(19) United States
(12) Patent Application Publication
(10) Pub. No.: US 2026/0325457 A1
(43) Pub. Date: Oct. 22, 2026

(54) LIPID NANOPARTICLE COMPOSITIONS AND METHODS OF USING SAME

(71) Applicants: Children's Hospital Medical Center, Cincinnati, OH (US); University of Cincinnati, Cincinnati, OH (US)

(72) Inventors: Bingxin Liu, Cincinnati, OH (US); Yutaka Maeda, Cincinnati, OH (US); Jeffrey Whitsett, Cincinnati, OH (US); Donglu Shi, Loveland, OH (US)

(21) Appl. No.: 19/650,603

(22) Filed: Apr. 17, 2026

Related U.S. Application Data
(60) Provisional application No. 63/790,898, filed on Apr. 18, 2025.

(51) Int. Cl. A61K 9/51; A61K 31/431; A61K 31/519

(52) U.S. Cl. CPC A61K 9/5123; A61K 31/431; A61K 31/519

(57) ABSTRACT

The present disclosure relates to water-soluble lipid nanoparticle (LNP) compositions comprising a hydrophobic small molecule encapsulated in an LNP that can be dispersed in an aqueous carrier. In certain aspects, the LNP comprises dipalmitoylphosphatidylcholine (DPPC), 1,2-dioleoyl-3-trimethylammonium-propane (DOTAP), distearoylphosphatidylethanolamine-polyethylene glycol (DSPE-PEG), and cholesterol. The LNP compositions can be used to encapsulate the hydrophobic small molecule 3-[2-[(4-fluorophenyl)methyl]imidazo[2,1-b]thiazol-6-yl]-2H-1-benzopyran-2-one (iMDK), an inhibitor of midkine (MDK) for treatment. The LNP compositions can be administered to treat a cancer in an individual in need thereof.

### CROSS-REFERENCE TO RELATED APPLICATIONS

The present patent application claims priority to U.S. Provisional Application No. 63/790,898, filed Apr. 18, 2025, which is incorporated herein by reference in its entirety.

### STATEMENT REGARDING FEDERALLY-SPONSORED RESEARCH

This invention was made with government support under HL134745, HL148856, HL164414, CA240317, and HL147351 awarded by the National Institutes of Health. The government has certain rights in the invention.

### BACKGROUND

A substantial number of small-molecule therapeutic agents under investigation are hydrophobic in nature and exhibit poor aqueous solubility. Such physicochemical properties complicate pharmaceutical formulation, limit bioavailability, and often necessitate the use of non-physiological solubilizing agents. These challenges have impeded the successful clinical translation of many otherwise promising therapeutic compounds and have constrained the effectiveness of current treatment regimens.

These formulation challenges are particularly acute in the context of cancer therapy. Various cancers such as oral squamous cell carcinoma, human prostate cancer, non-small cell lung cancer (NSCLC), lymphoma, and multiple myeloma continue to present significant clinical challenges, including poor patient outcomes, dose-limiting toxicities, and limited effectiveness of available therapies in certain patient populations. In many cases, disease heterogeneity, acquired resistance, and systemic toxicity limit the utility of existing small-molecule anticancer agents, underscoring the need for improved therapeutic formulations and delivery strategies.

Genetic alterations in oncogenic signaling pathways further complicate treatment. For example, cancers harboring mutations in the Kirsten rat sarcoma viral oncogene homolog (KRAS) are widely recognized as difficult to treat. KRAS-mutant cancers, including KRAS-mutant lung cancers, are often associated with aggressive disease, therapeutic resistance, and limited responsiveness to conventional chemotherapeutic agents. Although targeted therapies have been developed for select KRAS mutations, effective treatment options remain limited, and formulation-related challenges, such as hydrophobicity of small molecules, continue to restrict therapeutic efficacy and tolerability.

In an effort to address these formulation-related limitations, alternative formulations have been developed. For example, one such formulation, nanoparticle albumin-bound paclitaxel (nab-paclitaxel), encapsulates paclitaxel within albumin-based nanoparticles, enabling administration in an aqueous form without the use of Cremophor EL or ethanol. Nab-paclitaxel has been approved by the FDA and demonstrates reduced excipient-related toxicity. Nevertheless, albumin-based nanoparticle systems are not universally applicable, and challenges remain with respect to scalability, stability, and adaptability to other hydrophobic therapeutic agents, including agents targeting genetically defined cancer subtypes.

More recently, lipid nanoparticle (LNP) technologies have emerged as delivery systems for therapeutic agents. Lipid nanoparticles have demonstrated safety and effectiveness in delivering certain classes of therapeutic molecules, including nucleic acids such as messenger RNA (mRNA). However, existing LNP platforms have largely been optimized for nucleic acid delivery rather than hydrophobic small-molecule drugs. As such, there remains a need in the art for improved compositions and delivery systems capable of formulating and delivering hydrophobic small-molecule therapeutics, particularly those intended for the treatment of cancers with high unmet medical need, including but not limited to KRAS-mutant cancers, while minimizing toxicity and improving therapeutic outcomes.

### BRIEF SUMMARY

The present disclosure relates to lipid nanoparticle (LNP) compositions suitable for aqueous formulation and useful for encapsulating a hydrophobic small molecule for administration to an individual in need thereof. In aspects, the LNP composition comprises DPPC, DOTAP, DSPE-PEG, and cholesterol. In certain embodiments, the LNP composition further comprises a hydrophobic small molecule encapsulated therein. In aspects, the aqueous formulations of the present disclosure reduce or eliminate the need for toxic organic solvents, such as DMSO, that are otherwise required to solubilize hydrophobic small molecules for administration. In certain embodiments, the hydrophobic small molecule is iMDK, a small molecule inhibitor of Midkine (MDK).

Also disclosed are methods of treating a cancer in an individual in need thereof, comprising administering to the individual a therapeutically effective amount of an LNP composition of the present disclosure comprising iMDK. In certain embodiments, the cancer is a KRAS-mutant cancer, including a KRAS-mutant lung cancer. In certain embodiments, the cancer is resistant to a KRAS inhibitor, including sotorasib. In certain embodiments, the cancer is characterized by retention or activity of the ATF3-CHOP pathway. In aspects, the individual in need thereof has been identified as having one or more of the foregoing cancer characteristics prior to administration.

### BRIEF DESCRIPTION OF THE DRAWINGS

This application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1 depicts the iMDK structure (top) and a schematic of a lipid nanoparticle (LNP) encapsulating iMDK for inducing apoptosis in a cancer cell.

FIG. 2 depicts the following: iMDK suppressed the growth of KRASG12C inhibitor (sotorasib)-resistant H358 cells (H358R) carrying KRASG12C. (a) Phase contrast images showing H358 and H358R cells that were treated with DMSO, sotorasib (10 μM) or iMDK (5 μM) for 48 h. (b) Cell viability measured by MTS assay for H358 and H358R cells that were treated with DMSO, sotorasib (10 μM) or iMDK (5 μM) for 48 h (n=6). (c) Immunoblots indicating the expression of proteins from cell extracts of H358 and H358R cells that were treated with DMSO, sotorasib (10 μM) or iMDK (5 μM) for 48 h. (d) Immunoblots indicating the expression of proteins from cell extracts of H358 and H358R cells that were treated with DMSO or miransertib (10 μM) for 48 h. (e) Proposed mechanism by which iMDK induces apoptosis through induction of ATF3. Student's t-test values are indicated above bars. Error bars represent±standard deviation.

FIG. 3 depicts lipid nanoparticles (LNP; DPPC/DOTAP/DSPE-PEG/Cholesterol) carrying iMDK induce apoptosis in H441 cells in vitro. (a) Experimental scheme synthesizing LNP carrying iMDK. (b) Structure of lipid (left) or PEI (right) nanoparticles. (c) Zeta potential of DPPC/DOTAP/DSPE-PEG/Cholesterol (hereafter, LNP[DOTAP]), DPPC/DPPG/DSPE-PEG/Cholesterol (hereafter, LNP[DPPG]), and PEI-PEG-LinA that carry iMDK (n=3). (d) Hydrodynamic diameters (size) of LNP[DOTAP], LNP[DPPG], and PEI-PEG-LinA that carry iMDK (n=3). (e) Cell viability measured by MTS assay for H441 cells that were treated with LNP[DOTAP], LNP[DPPG], and PEI-PEG-LinA that carry iMDK estimated at 5 μM for 48 h (n=8). (f) Immunoblots indicating the expression of proteins from cell extracts of H441 cells that were treated with LNP[DOTAP], LNP[DPPG], and PEI-PEG-LinA that carry iMDK estimated at 5 μM for 48 h. Panels a and b were made with BioRender.com. Student's t-test values are indicated above bars. Error bars represent±standard deviation.

FIG. 4 depicts lipid nanoparticles (LNP; DPPC/DOTAP/DSPE-PEG/Cholesterol) carrying iMDK (iMDK-LNP) induce apoptosis in H441 xenografts without causing liver toxicity. (a) Shown are H441 xenografts from nude mice that were intraperitoneally treated with iMDK dissolved in DMSO (iMDK-DMSO) or iMDK-LNP at 9.7 mg/kg body weight once daily for 13 days. (b) Tumor volume of H441 xenografts that were untreated (n=7) or treated with iMDK-DMSO (n=8) or iMDK-LNP (n=8), as described in (a). P values comparing day-13 tumor volumes are shown. (c) Morphology and the expression of MDK and cleaved PARP in H441 xenografts from (a,b) are shown by H&E and immunohistochemical staining. Bars=100 μm. (d) Cleaved PARP1-positive cells (%) in H441 xenografts from (c) (n=3). (e) Body weight of nude mice with H441 xenografts described in (b). (f) ALT activity from serum of nude mice with H441 xenografts that were untreated or treated with iMDK-DMSO or iMDK-LNP as described in (a) for 13 days (n=3). All P values are from Student's t-test, as indicated. Error bars represent±standard deviation.

### DETAILED DESCRIPTION

### Definitions

Unless otherwise noted, terms are to be understood according to conventional usage by those of ordinary skill in the relevant art. In case of conflict, the present document, including definitions, will control. Exemplary methods and materials are described below, although methods and materials similar or equivalent to those described herein may be used in practice or testing of the present invention. All publications, patent applications, patents and other references mentioned herein are incorporated by reference in their entirety. The materials, methods, and examples disclosed herein are illustrative only and not intended to be limiting. The methods may comprise, consist of, or consist essentially of the elements of the compositions and/or methods as described herein, as well as any additional or optional element described herein or otherwise useful in the use or manufacture of LNP compositions comprising a hydrophobic small molecule, or their use in the treatment of a cancer as described herein.

As used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” may mean within 1 or more than 1 standard deviation, per the practice in the art. Alternatively, “about” may mean a range of up to 20%, or up to 10%, or up to 5%, or up to 1% of a given value. Alternatively, particularly with respect to biological systems or processes, the term may mean within an order of magnitude, such as within 5-fold, or such as within 2-fold, of a value. Where particular values are described in the application and claims, unless otherwise stated the term “about” meaning within an acceptable error range for the particular value should be assumed.

As used herein, the term “effective amount” means the amount of one or more active components that is sufficient to show a desired effect. This includes both therapeutic and prophylactic effects. When applied to an individual active ingredient, administered alone, the term refers to that ingredient alone. When applied to a combination, the term refers to combined amounts of the active ingredients that result in the therapeutic effect, whether administered in combination, serially or simultaneously.

The terms “individual,” “host,” “subject,” and “patient” are used interchangeably to refer to an animal that is the object of treatment, observation, and/or experiment. Generally, the term refers to a human patient, but the methods and compositions may be equally applicable to non-human subjects such as other mammals. In some aspects, the terms refer to humans. In further aspects, the terms may refer to a child (e.g., a pre-pubescent individual).

As used herein, the term “lipid nanoparticle” or “LNP” refers to a particle, vesicle, or other lipid-based assembly comprising one or more lipids and having a hydrophobic lipid region and a hydrophilic outer surface, and includes liposomes and other lipid vesicles, micellar structures, and lipid-based nanoparticles capable of associating with and/or encapsulating a therapeutic agent. Unless otherwise indicated, an LNP as described herein may optionally comprise one or more phospholipids, cationic lipids, PEGylated lipids, sterols (e.g., cholesterol), and combinations thereof.

As used herein, “water-soluble,” “aqueous,” or “soluble in water,” when used in reference to an LNP composition, means that the composition is capable of being formulated in an aqueous carrier (e.g., water, saline, or a buffered aqueous solution) to provide a flowable dispersion suitable for administration without requiring a toxic organic solvent as the primary carrier. In certain embodiments, the aqueous formulation is visually uniform (i.e., free of visible precipitate) after gentle mixing at room temperature for a period of time sufficient for administration.

As used herein, “encapsulated,” “encapsulation,” or “loaded” means that a therapeutic agent (e.g., iMDK) is incorporated within an LNP (e.g., within a hydrophobic region and/or interior of the LNP) such that the agent is carried by the LNP in an aqueous medium. Encapsulation may include complete or partial encapsulation. In certain embodiments, “encapsulated” refers to an association that is retained following one or more purification steps (e.g., dialysis) intended to remove unencapsulated (free) agent.

As used herein, “hydrophobic,” when used to describe a small molecule, refers to a compound having low solubility in water and a tendency to partition into a nonpolar phase relative to water. In certain embodiments, a hydrophobic small molecule cannot be formulated for administration in an aqueous carrier without a solubilizing aid (e.g., a surfactant and/or organic solvent) and/or that precipitates from an aqueous carrier at a therapeutically relevant concentration.

As used herein, “therapeutically effective amount” means an amount of a therapeutic agent (e.g., iMDK, including iMDK encapsulated in an LNP) that, when administered to an individual, is sufficient to treat a disease or condition (e.g., cancer), including to produce one or more beneficial therapeutic effects. A therapeutically effective amount may vary depending on, for example, the specific cancer, the stage of disease, the route of administration, and characteristics of the individual.

As used herein, “essentially free of DMSO” means that DMSO is not intentionally added as a solvent or carrier for administration. In certain embodiments, “essentially free of DMSO” means the composition contains no more than a trace amount of DMSO (e.g., residual DMSO, if any, from a manufacturing process) that does not materially contribute to solubilization of the therapeutic agent.

As used herein, “particle size” in reference to an LNP refers to an apparent particle diameter. In certain embodiments, particle size refers to hydrodynamic diameter measured by dynamic light scattering (DLS) in an aqueous medium at about room temperature. As used herein, “surface charge” may be expressed as “zeta potential,” which refers to an electrokinetic potential measured for particles dispersed in an aqueous medium, for example using a zeta potential analyzer.

As used herein, “resistant” (e.g., “KRAS inhibitor-resistant” or “sotorasib-resistant”) refers to reduced sensitivity of a cancer or cancer cell to a referenced therapy. Resistance may be intrinsic or acquired. In certain embodiments, resistance refers to a lack of clinically meaningful response and/or disease progression despite treatment with the referenced therapy. In certain embodiments, resistance refers to a reduced response in an in vitro or in vivo model relative to a corresponding sensitive control.

As used herein, a cancer that “retains the ATF3-CHOP-mediated apoptotic pathway” refers to a cancer in which ATF3 and CHOP (DDIT3) signaling remains functional such that induction of the pathway can result in apoptosis. In certain embodiments, retention of the pathway refers to the presence and inducibility of ATF3 and CHOP in cancer cells (e.g., following exposure to iMDK) and/or downstream apoptotic markers associated with pathway activation.

As used herein, “treat,” “treating,” or “treatment” of cancer refers to any detectable beneficial effect on a cancer in an individual, including but not limited to: inhibiting or reducing tumor growth, reducing tumor volume or burden, reducing or delaying metastasis, slowing or delaying disease progression, increasing progression-free survival, increasing overall survival, and/or reducing one or more symptoms associated with the cancer or its treatment.

### DETAILED DESCRIPTION

In one aspect, the lipid nanoparticle composition described herein comprises a hydrophobic small molecule encapsulated in a water-soluble lipid nanoparticle (LNP). In aspects, the LNP provides a hydrophilic outer surface that renders the encapsulated hydrophobic small molecule dispersible in an aqueous carrier, thereby enabling aqueous administration of molecules that would otherwise require toxic organic solvents such as dimethyl sulfoxide (DMSO). Lipid nanoparticle compositions and constituent lipids, as well as methods of making lipid nanoparticle compositions, are known in the art. Examples of LNP compositions and methods of synthesis are described, for example, in WO2025061874 (Lindner et al.) and US20250319037 (Toyonaga et al.).

In one aspect, the water-soluble LNP comprises one or more of dipalmitoylphosphatidylcholine (DPPC), 1,2-dioleoyl-3-trimethylammonium-propane (DOTAP), distearoylphosphatidylethanolamine-polyethylene glycol (DSPE-PEG), and cholesterol. In further aspects, the LNP comprises DPPC, DOTAP, DSPE-PEG, and cholesterol. In some embodiments, DPPC, DOTAP, DSPE-PEG, and cholesterol are present in a weight ratio of about 10:5:5:2, respectively. In other embodiments, the ratio of DPPC:DOTAP:DSPE-PEG:cholesterol is within a range from about 8:4:4:1.5 to about 12:6:6:2.5. It will be understood that the foregoing ratios are exemplary and that the skilled artisan may adjust individual component amounts while retaining the functional properties of the LNP compositions described herein.

In one aspect, the LNP composition that is dispersible in an aqueous carrier comprises a structural phospholipid. In certain embodiments, the structural phospholipid comprises dipalmitoylphosphatidylcholine (DPPC), also referred to as 16:0 PC. Without wishing to be bound by theory, DPPC may contribute to formation of a bilayer structure of the LNP.

In one aspect, the LNP composition that is dispersible in an aqueous carrier comprises a cationic lipid. In certain embodiments, the cationic lipid comprises 1,2-dioleoyl-3-trimethylammonium-propane (DOTAP), also referred to as 18:1 TAP. Without wishing to be bound by theory, inclusion of a cationic lipid such as DOTAP may impart a positive surface charge that can facilitate interaction of the LNP with target cells.

In one aspect, the LNP composition that is dispersible in an aqueous carrier comprises a PEGylated lipid component. In certain embodiments, the PEGylated lipid comprises distearoylphosphatidylethanolamine-polyethylene glycol (DSPE-PEG), for example DSPE-PEG 2000. Without wishing to be bound by theory, a PEGylated lipid may contribute to the hydrophilic outer surface of the LNP, promote colloidal stability, and/or enhance in vivo circulation time.

In one aspect, the LNP composition that is dispersible in an aqueous carrier comprises a structural lipid comprising cholesterol. Without wishing to be bound by theory, cholesterol may stabilize the LNP structure (e.g., by modulating membrane fluidity and/or rigidity). In certain embodiments, cholesterol is present in an amount effective to promote LNP stability while maintaining a desired surface charge and/or payload-loading performance.

In one aspect, the LNP composition encapsulates a hydrophobic small molecule within a hydrophobic region of the LNP. In certain embodiments, the hydrophobic small molecule comprises iMDK (3-[2-[(4-Fluorophenyl)methyl]imidazo[2,1-b]thiazol-6-yl]-2H-1-benzopyran-2-one). In its free form, iMDK exhibits low aqueous solubility; in certain embodiments, encapsulation in the LNP enables iMDK to be formulated in an aqueous carrier for administration.

In one aspect, a method of preparing the LNP composition comprises dissolving the lipid components in an organic solvent, removing the organic solvent to form a lipid film, hydrating the lipid film with deionized water to form liposomes, sonicating the liposome suspension to reduce particle size and promote uniformity, adding iMDK dissolved in tetrahydrofuran (THF) dropwise to the liposome suspension under sonication, and removing THF by rotary evaporation followed by dialysis against deionized water to remove residual THF and unencapsulated iMDK. In aspects, the lipid components are dissolved in chloroform at concentrations of about 25 mg/mL (DPPC), about 12.5 mg/mL (DOTAP), about 12.5 mg/mL (DSPE-PEG), and about 5 mg/mL (cholesterol). In aspects, iMDK is dissolved in THF at a concentration of about 5 mg/mL prior to addition to the liposome suspension. In aspects, the LNP composition is sonicated prior to use to ensure uniformity.

In further aspects, the LNP composition has a positive surface charge. In aspects, the surface charge of the LNP composition comprising DPPC, DOTAP, DSPE-PEG, and cholesterol is approximately 28 mV as measured by zeta potential analysis. Without wishing to be bound by theory, this positive surface charge facilitates interaction of the LNP with the negatively charged cell surface, promoting cellular uptake and delivery of the encapsulated hydrophobic small molecule into target cells.

In further aspects, the LNP composition has a uniform particle size distribution. In aspects, the size distribution of the LNP composition comprising DPPC, DOTAP, DSPE-PEG, and cholesterol is uniformly distributed at approximately 200 nm as measured by dynamic light scattering. In some aspects, the LNP composition has a particle size of about 100 nm to about 300 nm. In further aspects, the LNP composition has a particle size of about 150 nm to about 250 nm. In certain aspects, the LNP composition has a particle size of about 200 nm. The uniform size distribution of the LNP composition is understood to be a property conducive to efficient cellular delivery of the encapsulated small molecule.

In aspects, the LNP composition comprising DPPC, DOTAP, DSPE-PEG, and cholesterol encapsulating iMDK is capable of inducing apoptosis in KRAS-mutant cancer cells in vitro and suppressing tumor growth in vivo following intraperitoneal administration, without inducing liver toxicity as assessed by serum alanine aminotransferase (ALT) activity, in contrast to iMDK administered in DMSO. In aspects, a hydrophobic small molecule that exhibits low aqueous solubility is encapsulated in the LNP. Encapsulation of the hydrophobic small molecule in the LNP compositions described herein may enable the hydrophobic small molecule to be formulated in an aqueous carrier (e.g., as a flowable dispersion) for administration to an individual in need thereof.

In one aspect, the hydrophobic small molecule encapsulated in the LNP composition is the hydrophobic small molecule 3-[2-[(4-Fluorophenyl)methyl]imidazo[2,1-b]thiazol-6-yl]-2H-1-benzopyran-2-one (“iMDK”). In this aspect, the encapsulated iMDK can be formulated in an aqueous carrier for administration to an individual in need thereof, as further described herein.

### Pharmaceutical Compositions

In a further aspect, disclosed are pharmaceutical compositions comprising the LNP composition described herein. In one aspect, the pharmaceutical composition comprises a hydrophobic small molecule encapsulated in an LNP comprising DOTAP, wherein the composition is capable of being formulated in an aqueous carrier as described herein. In a further aspect, the pharmaceutical composition comprises a hydrophobic small molecule encapsulated in an LNP comprising one or more of dipalmitoylphosphatidylcholine (DPPC), DOTAP, distearoylphosphatidylethanolamine-polyethylene glycol (DSPE-PEG), and cholesterol, wherein the composition is capable of being formulated in an aqueous carrier as described herein. In further aspects, the pharmaceutical composition comprises an LNP comprising DPPC, DOTAP, DSPE-PEG, and cholesterol. In some aspects, DPPC, DOTAP, DSPE-PEG, and cholesterol are present in a weight ratio of about 10:5:5:2. In further aspects, the weight ratio of DPPC:DOTAP:DSPE-PEG:cholesterol is about 10:5:5:2.

As used herein with respect to the pharmaceutical compositions, the hydrophobic small molecule encapsulated in the LNP is a hydrophobic small molecule administered for a therapeutic or prophylactic effect upon administration to an individual in need thereof. The term “small molecule” is generally understood to one of ordinary skill in the art. As used herein, a “small molecule” refers to a molecule having a molecular weight of less than about 900 Da, which is non-biologic (e.g., not an antibody, protein, or nucleic acid), and which is chemically synthesized (in contrast to a biological molecule that is produced in a cell). Suitable hydrophobic small molecules that may be used with the disclosed LNPs will be readily appreciated by one of ordinary skill in the art, and include small molecules which are in general hydrophobic and which elicit a desirable effect upon administration to an individual. In one aspect, the hydrophobic small molecule is iMDK.

The pharmaceutical compositions of the present disclosure may further comprise one or more pharmaceutically acceptable excipients. Suitable excipients include, but are not limited to, aqueous buffers, tonicity agents, cryoprotectants, lyoprotectants, antioxidants, and pH-adjusting agents. Aqueous buffers useful in the compositions include phosphate-buffered saline (PBS), citrate buffer, acetate buffer, HEPES buffer, and Tris-HCl buffer, and are typically employed at a pH of about 6.5 to about 7.5. Tonicity agents, such as sodium chloride, sucrose, trehalose, or mannitol, may be included to render the composition isotonic and suitable for parenteral administration. Antioxidants, including but not limited to alpha-tocopherol, ascorbic acid, and butylated hydroxytoluene (BHT), may optionally be incorporated to minimize oxidative degradation of lipid components during storage.

The pharmaceutical compositions may further comprise cryoprotectants or lyoprotectants, such as sucrose, trehalose, lactose, or mannitol, to stabilize the LNP structure during lyophilization and/or frozen storage and upon reconstitution. In certain embodiments, the compositions may include one or more surfactants, such as polysorbate 80 (Tween 80) or poloxamer 188, to maintain colloidal stability and reduce aggregation. Chelating agents, such as ethylenediaminetetraacetic acid (EDTA), may optionally be included to sequester divalent metal ions that could otherwise promote lipid oxidation or destabilize the nanoparticle formulation. The skilled artisan will appreciate that the selection, concentration, and combination of excipients will depend on the intended route of administration, desired shelf life, storage conditions, and the physicochemical properties of the encapsulated hydrophobic small molecule.

### Method of Treating Cancer

The disclosed LNP compositions can be used to deliver hydrophobic small molecules to a cell for treatment of a disease state.

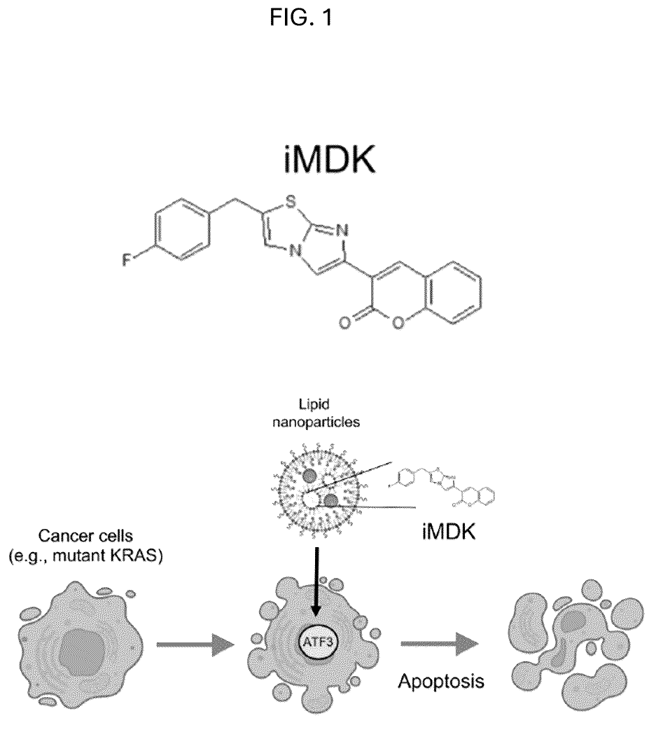

In particular, disclosed herein are methods of treating certain cancers using 3-[2-[(4-Fluorophenyl)methyl]imidazo[2,1-b]thiazol-6-yl]-2H-1-benzopyran-2-one, also referred to herein as “iMDK.” iMDK is a hydrophobic small molecule that exhibits low aqueous solubility, which can limit its translation into the clinic. iMDK is described in, for example, U.S. Patent Publication 20180177768, to Whitsett and Maeda, entitled “Methods and compositions to treat cancer” and U.S. Patent Publication 20210315903, entitled “Methods and compositions to treat cancer.” The disclosures of each are incorporated herein in their entirety, for all purposes. The structure of iMDK is shown in FIG. 1. FIG. 1 further depicts an exemplary schematic of treatment of a cell using an LNP containing iMDK. In the case of cancer, delivery of iMDK to a cancer cell may induce apoptosis, thereby targeting cancer cells and reducing disease burden. iMDK inhibits expression of MDK (also referred to as midkine), a growth factor. In certain embodiments, iMDK inhibits the PI3K-AKT pathway and induces apoptosis in various cell lines, for example HEK293 human embryonic kidney cells and H520 human lung squamous cell carcinoma cells. Notably, in certain embodiments, iMDK does not induce apoptosis in A549 human lung carcinoma cells that carry KRAS^{G12S} or in NHLF normal human lung fibroblasts.

Thus, in one aspect, disclosed herein are methods for treating a disease state by administering to an individual in need thereof a lipid nanoparticle (LNP) comprising iMDK, wherein the disease state is cancer. In aspects, the cancer retains the ATF3-mediated apoptotic pathway. In aspects, the cancer is a KRAS-mutant cancer. In further aspects, the cancer is a KRAS-mutant lung cancer. In a yet further aspect, the cancer is a lung cancer characterized by a carcinoma having the driver oncogene KRAS^{G12V}. In a yet further aspect, the cancer is a papillary lung adenocarcinoma having the driver oncogene KRAS^{G12V}. In a yet further aspect, the cancer is characterized by a carcinoma having the driver oncogene KRAS^{G12C}. In a yet further aspect, the cancer is a bronchioalveolar carcinoma having the driver oncogene KRAS^{G12C}. In a yet further aspect, the cancer is non-small cell lung cancer (NSCLC) that harbors KRAS^{G12C}. In a yet further aspect, the cancer is a sotorasib-resistant cancer. In a yet further aspect, the cancer is a sotorasib-resistant cancer that retains the ATF3-mediated apoptotic pathway. In a yet further aspect, the cancer is a sotorasib-resistant KRAS^{G12C}-mutant cancer. In a yet further aspect, the cancer is sotorasib-resistant H358 bronchioalveolar carcinoma comprising cells that harbor KRAS^{G12C}. In a yet further aspect, the cancer is a cancer that does not respond to inhibition of the PI3K-AKT pathway. In a yet further aspect, the cancer is a KRAS inhibitor-resistant cancer that does not respond to PI3K-AKT inhibition but retains the ATF3-mediated apoptotic pathway. In aspects, the cancer is selected from oral squamous cell carcinoma, prostate cancer, primary effusion lymphoma, and multiple myeloma. In a yet further aspect, the cancer is multiple myeloma, wherein iMDK-mediated apoptosis proceeds through activation of the ATF3-CHOP endoplasmic reticulum stress pathway.

In a further aspect, the method comprises: (a) identifying an individual likely to respond to iMDK; and (b) administering an LNP comprising iMDK to the individual.

In one aspect, identifying an individual likely to respond to iMDK comprises determining that the individual has a cancer that retains the ATF3-mediated apoptotic pathway. In one aspect, identifying an individual likely to respond to iMDK comprises determining that the individual has a cancer in which targeting the ATF3-CHOP endoplasmic reticulum stress pathway is likely to effect a positive therapeutic outcome. In one aspect, identifying an individual likely to respond to iMDK comprises determining that the individual has a KRAS-mutant cancer. In one aspect, identifying an individual likely to respond to iMDK comprises determining that the individual has a KRAS-mutant lung cancer. In one aspect, identifying an individual likely to respond to iMDK comprises determining that the individual has a carcinoma having the driver oncogene KRAS^{G12V}. In one aspect, identifying an individual likely to respond to iMDK comprises determining that the individual has a papillary lung adenocarcinoma having the driver oncogene KRAS^{G12V}. In one aspect, identifying an individual likely to respond to iMDK comprises determining that the individual has a carcinoma having the driver oncogene KRAS^{G12C}. In one aspect, identifying an individual likely to respond to iMDK comprises determining that the individual has a bronchioalveolar carcinoma having the driver oncogene KRAS^{G12C}. In one aspect, identifying an individual likely to respond to iMDK comprises determining that the individual has non-small cell lung cancer (NSCLC) harboring KRAS^{G12C}. In one aspect, identifying an individual likely to respond to iMDK comprises determining that the individual has a sotorasib-resistant cancer. In one aspect, identifying an individual likely to respond to iMDK comprises determining that the individual has a sotorasib-resistant cancer that retains the ATF3-mediated apoptotic pathway. In one aspect, identifying an individual likely to respond to iMDK comprises determining that the individual has a sotorasib-resistant KRAS^{G12C}-mutant cancer. In one aspect, identifying an individual likely to respond to iMDK comprises determining that the individual has sotorasib-resistant H358 bronchioalveolar carcinoma comprising cells that harbor KRAS^{G12C}. In one aspect, identifying an individual likely to respond to iMDK comprises determining that the individual has a cancer that does not respond to inhibition of the PI3K-AKT pathway. In one aspect, identifying an individual likely to respond to iMDK comprises determining that the individual has a KRAS inhibitor-resistant cancer that does not respond to PI3K-AKT inhibition but retains the ATF3-mediated apoptotic pathway. In one aspect, identifying an individual likely to respond to iMDK comprises determining that the individual has a cancer selected from oral squamous cell carcinoma, prostate cancer, primary effusion lymphoma, and multiple myeloma. In one aspect, identifying an individual likely to respond to iMDK comprises determining that the individual has multiple myeloma in which iMDK-mediated apoptosis is expected to proceed through activation of the ATF3-CHOP endoplasmic reticulum stress pathway.

### Methods of Administration

In aspects, the disclosed LNP compositions may be used to enable hydrophobic small molecules to be formulated in an aqueous carrier (e.g., as a flowable dispersion) for administration. The LNP compositions may provide reduced toxicity compared to conventional solvent-based delivery approaches. For example, hydrophobic small molecules encapsulated in the LNP compositions described herein may be administered without the use of toxic organic solvents such as dimethyl sulfoxide (DMSO) as the primary carrier. Thus, in aspects, the LNP compositions are essentially free of DMSO or a similarly toxic organic solvent. As set forth below, in aspects, the hydrophobic small molecule is iMDK, which may be delivered to an individual in need thereof.

In aspects, the LNP compositions described herein may be used to deliver hydrophobic small molecules to an individual in need thereof. For example, the LNP compositions may be useful for delivering therapeutic hydrophobic small molecules that are not eligible for albumin-bound nanoparticle formulations.

In aspects, an LNP composition comprising a therapeutic agent, such as a hydrophobic small molecule, may be used to deliver the hydrophobic small molecule intracellularly. In further aspects, the LNP composition may be used as a pharmaceutical composition for in vivo delivery of a hydrophobic small molecule. Via encapsulation within the LNP, the hydrophobic small molecule may be delivered to a target cell of an individual, such as, in certain embodiments, a cancer cell. In aspects, the LNP composition comprising a hydrophobic small molecule is administered to a subject in a pharmaceutically acceptable carrier, such as saline or a phosphate buffer. In aspects, the concentration of the lipid composition in a mixture with the pharmaceutically acceptable carrier is from about 0.05% by weight to about 90% by weight. In further aspects, one or more pharmaceutically acceptable additives such as a pH-adjusting buffer and/or an osmotic pressure adjustment agent may be added to the LNP composition.

In aspects, the route of administration for administering the LNP compositions described herein includes oral administration and parenteral administration. Non-limiting examples of parenteral administration include intra-articular administration, intravenous administration, intra-arterial administration, subcutaneous administration, intracutaneous administration, intravitreal administration, intraperitoneal administration, intramuscular administration, intravaginal administration, intravesical administration, intrathecal administration, pulmonary administration, rectal administration, colonic administration, buccal administration, nasal administration, intracisternal administration, and inhalation. Delivery may also be performed by local administration in vivo. In certain embodiments, the LNP composition is administered by direct injection into a diseased site.

The dosage form of the LNP compositions of the present disclosure is not limited to any particular form. For parenteral administration, the LNP compositions may be formulated as sterile injectable solutions or suspensions, and may incorporate one or more additives including antioxidants, buffers, bacteriostats, isotonicity agents, suspending agents, solubilizers, thickeners, stabilizers, and preservatives, as appropriate. For oral administration, the compositions may be formulated in combination with suitable excipients as tablets, troches, capsules, pills, suspensions, syrups, or other oral dosage forms, where the stability and bioavailability of the LNP under gastrointestinal conditions supports such administration.

### EXAMPLES

The following non-limiting examples are provided to further illustrate embodiments of the invention disclosed herein. It should be appreciated by those of skill in the art that the techniques disclosed in the examples that follow represent approaches that have been found to function well in the practice of the invention, and thus may be considered to constitute examples of modes for its practice. However, those of skill in the art should, in light of the present disclosure, appreciate that many changes may be made in the specific embodiments that are disclosed and still obtain a like or similar result without departing from the spirit and scope of the invention.

Applicant has developed a lipid nanoparticle composition comprising DPPC/DOTAP/DSPE-PEG/cholesterol. In one aspect, the lipid nanoparticle composition comprises DPPC/DOTAP/DSPE-PEG/cholesterol in a weight ratio of about 10:5:5:2. The lipid nanoparticle composition may be used to deliver a hydrophobic small molecule. In one aspect, the hydrophobic small molecule is an anti-cancer agent, for example iMDK. In certain aspects, iMDK encapsulated in the lipid nanoparticle composition is capable of being formulated in an aqueous carrier as described herein.

It was found that iMDK encapsulated in DPPC/DOTAP/DSPE-PEG/cholesterol lipid nanoparticles and administered intraperitoneally in vivo suppressed the growth of lung cancer xenografts (H441 papillary lung adenocarcinoma cells that harbor KRAS^{G12V}) in nude mice as effectively as iMDK dissolved in DMSO. iMDK encapsulated in the DPPC/DOTAP/DSPE-PEG/cholesterol lipid nanoparticles exhibited reduced liver toxicity in nude mice relative to iMDK dissolved in DMSO. The DPPC/DOTAP/DSPE-PEG/cholesterol lipid nanoparticles may be used to deliver therapeutic hydrophobic small molecules in a formulation that is capable of being formulated in an aqueous carrier, which is expected to reduce excipient-related toxicity relative to carriers such as castor oil and ethanol.

In other aspects, the lipid nanoparticle composition may be used to deliver other hydrophobic drugs. For example, other hydrophobic drugs may be encapsulated in the disclosed lipid nanoparticle compositions and formulated in an aqueous carrier for administration.

### Example 1

iMDK (3-[2-[(4-Fluorophenyl)methyl]imidazo[2,1-b]thiazol-6-yl]-2H-1-benzopyran-2-one) (FIG. 1, top) was originally discovered as a small molecule that inhibits the expression of MDK (also known as midkine), a growth factor, that is expressed in H441 human lung papillary adenocarcinoma cells that carry the driver oncogene KRASG12V. Molecular and biochemical analyses indicated that iMDK also inhibited the PI3K-AKT pathway and induced apoptosis in H441 cells, HEK293 human embryonic kidney cells and H520 human lung squamous cell carcinoma cells but not A549 human lung carcinoma cells that carry KRASG12S or NHLF normal human lung fibroblasts. 1 iMDK has also been shown to effectively suppress growth of HSC-2 human oral squamous carcinoma cells, PC3 human prostate cancer cells, H1299 human non-small cell lung carcinoma cells, BCBL-1, BC-1, GTD, TY-1 primary effusion lymphoma cells and KMM-1, NCI-H929, RPMI-8226 multiple myeloma cells. Although iMDK is potentially a promising cancer drug to suppress the growth of subtypes of cancer cells, the molecular mechanism(s) by which iMDK suppresses cancer cell growth remains unknown. Also, the low aqueous solubility of iMDK due to its hydrophobicity hampers efforts to translate iMDK into the clinic. Previous in vitro and in vivo xenograft studies were conducted by dissolving iMDK into the toxic solvent DMSO, which is not suitable for preclinical and clinical application.

The small molecule iMDK induces apoptosis in H441 papillary lung adenocarcinoma cells that harbor KRASG12V; however, the mechanism by which iMDK induces apoptosis is not fully understood. To investigate the molecular mechanism, Applicant conducted an RNA-seq analysis to identify genes that are regulated by iMDK in H441 cells. RNA-seq data analysis indicated that iMDK activated the ATF3-CHOP (DDIT3)-mediated apoptotic pathway. iMDK did not induce apoptosis in ATF3 or CHOP CRISPR knockout H441 cells, indicating that iMDK induced apoptosis through the ATF3-CHOP pathway. Notably, iMDK also activated the ATF3-CHOP pathway and induced apoptosis in both sotorasib (KRASG12C inhibitor)-naïve and resistant H358 bronchioalveolar carcinoma cells that harbor KRASG12C. To enable iMDK to be formulated in an aqueous carrier, Applicant encapsulated iMDK in multiple lipid or polymer nanoparticles capable of providing a flowable dispersion in an aqueous carrier. iMDK that was encapsulated in DPPC/DOTAP/DSPE-PEG/cholesterol lipid nanoparticles but not DPPC/DPPG/DSPE-PEG/cholesterol or PEI-PEG-LinA nanoparticles induced apoptosis in H441 cells in vitro. Intraperitoneal injection of iMDK lipid nanoparticles (DPPC/DOTAP/DSPE-PEG/cholesterol), formulated in an aqueous carrier, significantly reduced the growth of H441 xenografts without inducing liver toxicity in vivo. These results suggest that iMDK lipid nanoparticle delivery is effective for treating subtypes of KRAS-mutant lung cancers that retain the ATF3-CHOP-mediated apoptotic pathway.

In the present study, to understand the molecular mechanism by which iMDK induces apoptosis in H441 lung cancer cells carrying KRAS^{G12V}, RNA-seq was combined with a gene knockout approach using CRISPR/Cas9 to identify genes that are involved in apoptosis induced by iMDK. The discovery of small molecules (e.g., sotorasib) that bind to KRAS^{G12C} and suppress the growth of cancers driven by KRAS^{G12C} has informed treatment strategies for KRAS-mutant lung cancers; however, such cancers can become resistant to KRAS^{G12C} inhibitors by different mechanisms, urging the development of novel therapeutics to treat KRAS^{G12C} inhibitor-resistant cancers. Applicant sought to determine whether iMDK can suppress the growth of sotorasib-resistant H358 bronchioalveolar carcinoma cells that harbor KRAS^{G12C}. Additionally, to aid future clinical application, Applicant developed an iMDK formulation that is capable of being formulated in an aqueous carrier by using lipid nanoparticle (LNP) technology to encapsulate iMDK, thereby reducing reliance on DMSO as the primary carrier.

### MATERIALS AND METHODS

### Materials

Lipids were purchased from Avanti Polar Lipids (Alabaster, AL, USA) including 18:1 TAP(DOTAP) (cat #: 890890), 16:0 PC (DPPC) (cat #: 850355), and 16:0 PG (DPPG) (cat #: 840455). O-Methyl-O′-succinylpolyethylene glycol 2′000 (cat #: 17928), poly(ethyleneimine) solution (cat #: 408700), linoleic acid (cat #: L1376) and the ALT Activity Assay (cat #: MAK052) were purchased from Sigma-Aldrich (St. Louis, MO, USA). EDC (cat #: 22981) and N-hydroxysuccinimide (NHS) (cat #: 24500) were purchased from ThermoFisher (Waltham, MA). iMDK (cat #5126) and sotorasib (also known as AMG510; cat #7713) were purchased from Tocris Biosciences (Minneapolis, MN, USA). Miransertib (also known as ARQ092; cat #HY-19719) was purchased from MedChemExpress (Monmouth Junction, NJ, USA).

### Cells

H441 papillary lung adenocarcinoma cells, A549 lung carcinoma cells, and H358 bronchioalveolar carcinoma cells were obtained from ATCC (Manassas, VA, USA). H358R cells (sotorasib-resistant H358 cells) were generated by culturing H358 cells at 10 μM of sotorasib for 8 months. ATF3 or CHOP (DDIT3) knockout H441 cells were created using ATF3 CRISPR Plasmids (h) (cat #sc-416577, Santa Cruz Biotechnology, Dallas, TX, USA) or DDIT3/CHOP/GADD153 CRISPR Plasmids (h) (cat #sc-400051-KO-2, Santa Cruz Biotechnology).

### Immunoblot Analysis

Cells were lysed in ice-cold RIPA Lysis and Extraction Buffer (cat #: PI89900; ThermoFisher, Waltham, MA, USA). Cell lysates were cleared by centrifugation (30 min at 10,000×g at 4° C.) and protein concentration was determined using Direct Detect® Assay-free Cards (cat #: DDAC00010-GR; Millipore, Burlington, MA, USA). Equal amounts of protein (50 μg) were separated on an SDS-PAGE gel. The gel was electrophoretically transferred to a Hybond PVDF transfer membrane and incubated with primary and secondary antibodies. Antibody specific for actin (cat #: LMAB-C4) was obtained from Seven Hills Bioreagents (Cincinnati, OH, USA). Antibody specific for MDK (also known as midkine) was obtained from Abcam (cat #: ab52637, Waltham, MA, USA). Antibodies specific for P-AKT (cat #: 4060S), AKT (cat #: 9272S), Cleaved PARP (cat #: 5625S), P-ERK (MAPK T202/Y204, cat #: 9101S), ERK1/2 (cat #: 4695S), CHOP (cat #: 2895S) and ATF3 (cat #: 18665S) were obtained from Cell Signaling (Danvers, MA, USA).

### Cell Viability Assay

Cells were plated in 24-well plates at a density of 1×10⁵ cells and cultured at 37° C. for 24 hours. Medium was removed by aspiration and replaced with fresh culture medium containing iMDK (5 μM), miransertib (10 μM) or sotorasib (10 μM) that was dissolved in DMSO or iMDK that was encapsulated in water-soluble nanoparticles. Cells were treated for 48 hours before an MTS assay to determine cell viability based on the manufacturer's protocol (CellTiter 96 AQ Cell proliferation assay, cat #: G3582; Promega, Madison, WI, USA)

### RNA-Seq Analysis

For RNA-seq, RNA was extracted from H441 cells treated with DMSO or iMDK at 5 μM for 24 hours. RNA was sent to CCHMC DNA sequencing core (Cincinnati, OH) as described previously.²⁵ Quality assessment and pre-processing of RNA-seq reads were performed using FASTQC, Trim Galore and SAMtools. Reads were then aligned to the hg38 genome using Bowtie2. Low-quality alignments were removed using SAMtools. Potential PCR duplication reads were identified using Picard tool and excluded. Gene expression was counted using htseq-count. Differential expression analysis was performed using Bioconductor DESeq2 package.²⁶ Differential expression with at least a 1.5-fold change and false discovery rate <0.1 was considered significant. Volcano plot showing differential expression result was generated using Bioconductor EnhancedVolcano package. Gene ontology (GO) analysis was conducted using the ToppGene Suite bioinformatical tool (https://toppgene.cchmc.org).¹¹

qRT-PCR

RNA was extracted from H441 cells, H358 and A549 cells using QIAGEN RNeasy Mini Kit (cat #74104; QIAGEN, Germantown MD, USA). Following extraction, reverse transcription reactions were carried out with an iScript cDNA synthesis kit (cat #1708890; Bio-Rad. Hercules, California, USA) according to the provided manufacturer's instructions. The synthesized cDNA was used for quantitative PCR (qPCR) using a StepOnePlus Real-Time PCR System with specific TaqMan gene expression assay probes for ATF3 (cat #: 4331182; Hs00231069_m1), CHOP (also known as DDIT3; cat #: 4331182; Hs00358796_g1), TNF (cat #: 4331182; Hs00174128_m1) and 18S RNA (cat #: 4333760T) (ThermoFisher). Expression levels of ATF3, CHOP (DDIT3) and TNF were normalized using 18S RNA to control for variations in RNA input and cDNA synthesis efficiency. The resultant data were expressed as mean±standard deviation (SD) and analyzed statistically using the 2-tailed Student's t-test to determine significance. All statistical analyses were conducted using GraphPad Prism software, version 10 (GraphPad Software, Inc.)

### Nanoparticle Formulation

DPPC/DOTAP/DSPE-PEG/Cholesterol (DPPC [DOTAP]) or DPPC/DPPG/DSPE-PEG/Cholesterol (DPPC [DPPG]) was dissolved in chloroform at the concentration of 25 mg/mL, 12.5 mg/mL, 12.5 mg/mL and 5 mg/mL, respectively. Each lipid preparation (3 ml) was mixed thoroughly in a round-bottom flask. The organic solvent was then removed using a rotary evaporator to form a thin lipid film on the walls of the flask. The lipid film was hydrated with 16.5 mL of deionized (DI) water to form liposomes at a final concentration of 10 mg/ml. The liposome suspension was put into a sonicator water bath for 2 hours at room temperature to reduce the particle size and ensure uniformity. The liposome solution was transferred to a 50 mL tube and subjected to additional water bath sonication. While maintaining sonication, 6.6 mL of iMDK dissolved in tetrahydrofuran (THF) at a concentration of 5 mg/mL was added dropwise to the liposome suspension. After the addition was complete, the mixture was sonicated for another 30 minutes. The iMDK-loaded lipid nanoparticle (LNP) solution was then transferred back to the round-bottom flask, and THE was removed using rotary evaporation. To purify the iMDK-loaded LNPs, the solution was dialyzed against DI water for 6 hours to remove any remaining THF and unencapsulated iMDK. The final iMDK LNP solution was then ready for use. Before each use, the iMDK LNPs were sonicated for an additional 30 minutes to ensure quality.

For PEI-PEG-LinA synthesis, polyethyleneimine (PEI) was functionalized with biological fatty acids and polyethylene glycol (PEG) via amidation using EDC/NHS-mediated coupling, following a standard reaction scheme for all coupling reactions as previously described.²⁷ Briefly, 450 mg (0.25 mmol) PEI (MW 1800) was dissolved in 15 ml ethanol at 40° C. for 15 mins. Next, 150 mg (0.075 mmol) PEG-COOH, 350 mg (1.250 mmol) LinA, 316 mg (1.656 mmol) EDC and 238 mg (2.070 mmol) NHS were dissolved in 15.75 ml ethanol plus 1.57 ml 500 mM MES buffer and reacted at 40° C. for 15 mins. The two solutions were immediately mixed and allowed to react overnight at 40° C. After the reaction, ethanol was removed via rotary evaporation, and the product was resuspended in deionized water. The conjugated PEI was dialyzed against deionized water using a 20 kDa membrane for 4-5 days, extracted twice with diethyl ether, and lyophilized. Before use, the lyophilized polymers were suspended in 10 mM MOPS buffer (pH 7.4) and sonicated.

### Mouse Experiments

All animal experiments conducted were approved by the Institutional Animal Care and Use Committee (IACUC). Human H441 lung cancer xenografts were established in 6-8-wk-old female NU/J nude mice (cat #002019; Jackson Laboratory, Bar Harbor, Maine, USA) by subcutaneous (s.c.) injection of H441 cells (1×10⁶/50 μl) mixed with Matrigel® (cat #CB-40234A; BD Pharmingen, San Diego, CA) into the dorsal flank. The mice were randomly assigned into three groups (n=8 per group) 5-8 days after tumor inoculations. iMDK (90 μg) was intraperitoneally injected every day. Tumors were measured 5, 10 and 13 days after the first injection of iMDK, and tumor volume was calculated as a×b2×0.5, where a and b were large and small diameters, respectively. After treatment for 13 days, body weights were measured, and mice were euthanized. Xenografts were then removed and prepared for histological analyses. For analysis of serum ALT, blood was drawn from the heart of the mice 13 days after the first iMDK injection.

### Results

RNA-Seq Indicates that iMDK Activates the ATF3-CHOP-Mediated Apoptotic Pathway

Applicant previously identified small molecule iMDK as an inhibitor of the expression of MDK and an inducer of apoptosis in H441 papillary lung adenocarcinoma cells that harbor KRAS^{G12V}.¹ Since Applicant also identified that iMDK inhibited the PI3K-AKT pathway in H441 cells¹, Applicant hypothesized that iMDK induces apoptosis through inhibition of the PI3K-AKT pathway. However, miransertib (also known as ARQ092),¹⁰ another PI3K-AKT pathway inhibitor, inhibited the PI3K-AKT pathway in H441 cells but failed to induce PARP cleavage, a marker for apoptosis, and failed to suppress cell growth, indicating that the inhibition of the PI3K-AKT pathway is not sufficient to explain the mechanism by which iMDK induced apoptosis in H441 cells. To identify the mechanism, Applicant sought to determine the genes that were affected by iMDK in an unbiased fashion using RNA-seq with RNA isolated from H441 cells that were treated with iMDK or DMSO (control) for 24 hours. RNA-seq analysis indicated that iMDK significantly induced 545 genes while reducing 491 genes (data not shown). Applicant analyzed the 545 genes induced by iMDK using the ToppGene Suite bioinformatics tool¹¹ (https://toppgene.cchmc.org), which indicated that 51 genes fall into positive regulation of apoptotic process (GO: 0043065). Among the 51 genes, ATF3, DDIT3 (also known as CHOP; hereafter, CHOP) and TNF are of particular interest since the ATF3-CHOP endoplasmic reticulum (ER) stress complex (GO: 1990622)¹² and TNF¹³ are known to induce apoptosis in specific cell types. The induction of ATF3, CHOP and TNF by iMDK in H441 cells was confirmed by TaqMan gene expression analysis and western blotting. Of note, miransertib neither induced the expression of ATF3 nor CHOP, indicating that the PI3K-AKT pathway does not mediate the induction of ATF3 and CHOP by iMDK. Previously, Applicant showed that iMDK did not induce apoptosis in A549 lung carcinoma cells that harbor KRAS^{G12S}.¹ Applicant assessed the expression of ATF3, CHOP and TNF in A549 cells treated with iMDK. Notably, iMDK induced the expression of TNF but not ATF3 or CHOP in A549 cells, suggesting that the induction of ATF3 and CHOP but not TNF by iMDK is required for iMDK-mediated induction of apoptosis in H441 cells.

ATF3-CHOP Complex is Required for the Apoptosis Induced by iMDK in H441 Cells

To determine the role of the ATF3-CHOP complex in iMDK-mediated apoptosis in H441 cells, Applicant used the CRISPR/Cas9 genome editor to knockout ATF3 or CHOP in H441 cells and assessed the apoptotic effect of iMDK in the knockout cells. Notably, iMDK induced cleaved PARP and reduced cell growth in parent H441 cells but not in ATF3 knockout H441 cells (two independent clones #2 and #9), indicating that iMDK requires ATF3 to induce apoptosis in H441 cells. Likewise, iMDK failed to induce cleaved PARP and reduce cell growth in CHOP knockout H441 cells (two independent clones #2 and #3), indicating that iMDK also requires CHOP to induce apoptosis in H441 cells. These data indicate that iMDK induces apoptosis in H441 cells through induction of the ATF3-CHOP complex.

iMDK Induces Apoptosis in KRAS^{G12C} Inhibitor (Sotorasib)-Naïve and Resistant H358 Bronchioalveolar Carcinoma Cells that Harbor KRAS^{G12C}

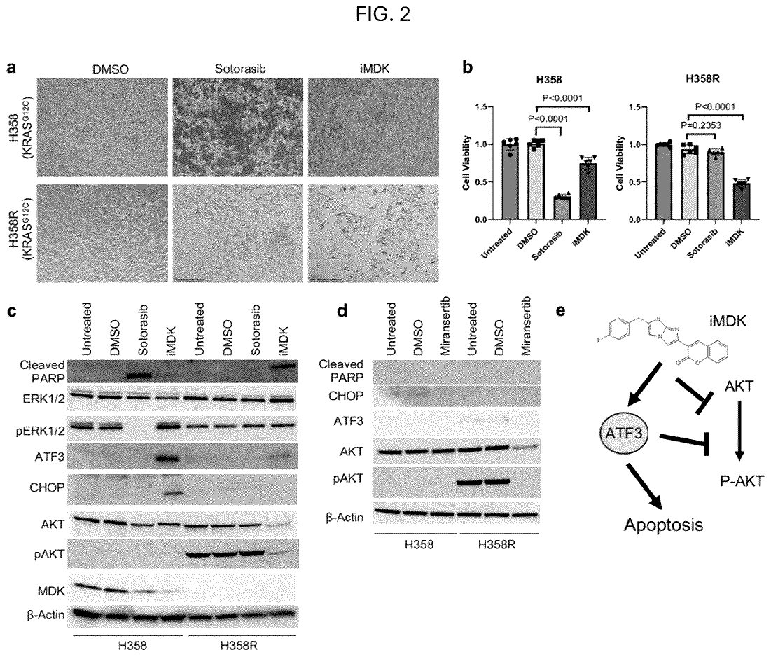

Sotorasib (also known as AMG510) has recently been approved¹⁴ by the FDA to treat non-small cell lung cancer (NSCLC) that harbors KRAS^{G12C}; however, the majority of the sotorasib-treated NSCLC recurs even after an initial successful response, therefore new therapeutics are required to target such sotorasib-resistant NSCLC. Applicant hypothesized that iMDK might suppress the growth of sotorasib-resistant NSCLC. Applicant created sotorasib-resistant H358 bronchioalveolar carcinoma cells that harbor KRAS^{G12C} by culturing the cells long term with sotorasib at 10 μM for 8 months (hereafter, H358R cells). We treated parent H358 and H358R cells with sotorasib or iMDK and assessed cell growth. As expected, sotorasib significantly suppressed the growth of H358 cells associated with the induction of cleaved PARP and inhibition of pERK but did not suppress the growth of H358R cells (FIG. 2, panels a, b and c). iMDK also significantly suppressed the growth of H358 cells associated with induction of cleaved PARP, ATF3 and CHOP though less effectively than sotorasib (of note, the expression of pAKT was low in H358 cells) (FIG. 2, panels a, b and c). Importantly, iMDK significantly suppressed the growth of H358R cells associated with induction of cleaved PARP, ATF3 and CHOP as well as the inhibition of pAKT (FIG. 2, panels a, b and c). Miransertib, a PI3K-AKT pathway inhibitor, failed to induce cleaved PARP, an apoptosis marker, while it inhibited pAKT in H358R cells (FIG. 2, panel d). These results suggest that iMDK induces apoptosis in sotorasib-resistant H358R cells through the induction of ATF3-CHOP-mediated apoptosis but not by PI3K-AKT pathway inhibition (FIG. 2, panel e).

iMDK Encapsulated in Lipid Nanoparticles (LNP; DPPC/DOTAP/DSPE-PEG/Cholesterol) is Capable of being Formulated in an Aqueous Carrier and Induces Apoptosis in H441 Cells In Vitro

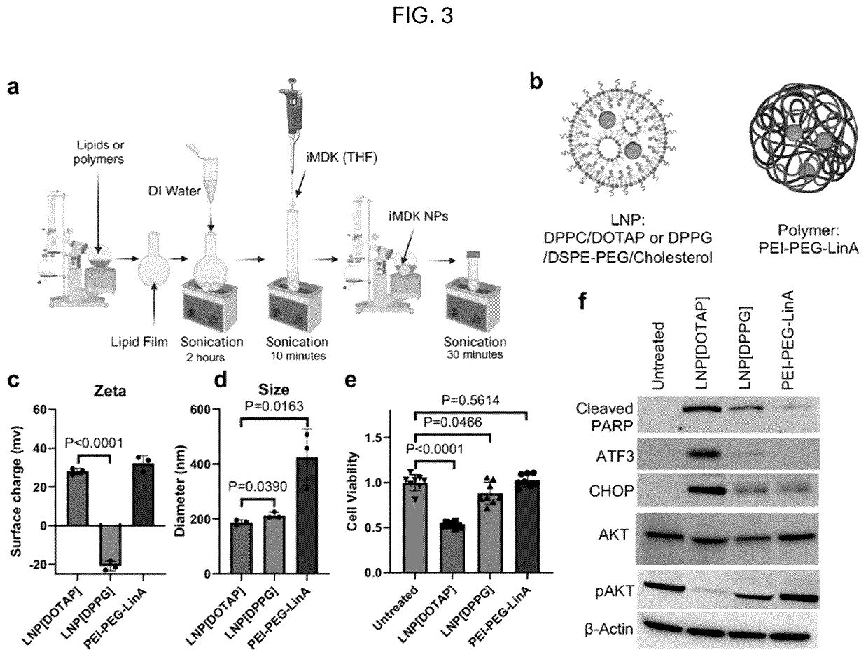

Due to its hydrophobic structure (FIG. 1, top), iMDK exhibits low aqueous solubility, which hampers efforts to translate iMDK into the clinic. To enable iMDK to be formulated in an aqueous carrier, Applicant employed nanoparticle systems that can encapsulate hydrophobic small molecules within a hydrophobic region while presenting a hydrophilic outer surface. Applicant tested three nanoparticle formulations as possible vehicles for encapsulating and delivering iMDK in an aqueous carrier: 1) DPPC/DOTAP/DSPE-PEG/cholesterol (lipid), 2) DPPC/DPPG/DSPE-PEG/cholesterol (lipid), and 3) PEI-PEG-LinA (polymer). Applicant mixed iMDK dissolved in tetrahydrofuran (THF), an organic solvent, with these three different nanoparticle formulations dispersed in an aqueous carrier. THF was removed from iMDK-nanoparticle mixtures by rotary evaporation followed by dialysis against water (FIG. 3. panels a and b), as described in the Methods section. Applicant measured the surface charge and size of these iMDK-encapsulating nanoparticle formulations in an aqueous carrier using a Zetasizer, since positively charged and uniformly distributed nanoparticles can facilitate delivery of small molecules into cancer cells. 15 The surface charges of DPPC/DOTAP/DSPE-PEG/cholesterol (hereafter, LNP[DOTAP]), DPPC/DPPG/DSPE-PEG/cholesterol (hereafter, LNP[DPPG]), and PEI-PEG-LinA were 28 mV, −20.9 mV, and 32 mV, respectively (FIG. 3, panel c), indicating that LNP[DOTAP] and PEI-PEG-LinA are positively charged while LNP[DPPG] is negatively charged. The size distribution of LNP[DOTAP] and LNP[DPPG] was uniformly distributed around 200 nm, while that of PEI-PEG-LinA was not uniformly distributed around 400 nm (data not shown). Applicant then treated H441 cells with these three different nanoparticle formulations encapsulating iMDK and assessed cell viability, the ATF3-CHOP pathway, and PI3K-AKT pathway activities. Notably, LNP[DOTAP] containing iMDK significantly inhibited cell viability associated with induction of the ATF3-CHOP pathway and inhibition of the PI3K-AKT pathway in H441 cells, while the other nanoparticle formulations (LNP[DPPG] and PEI-PEG-LinA) did not (FIG. 3, panels e and f), indicating that the LNP[DOTAP] formulation is suitable for delivering iMDK into H441 cells in vitro.

DPPC/DOTAP/DSPE-PEG/Cholesterol Nanoparticles (LNP[DOTAP]) Encapsulating iMDK Suppress the Growth of H441 Cell Xenografts In Vivo

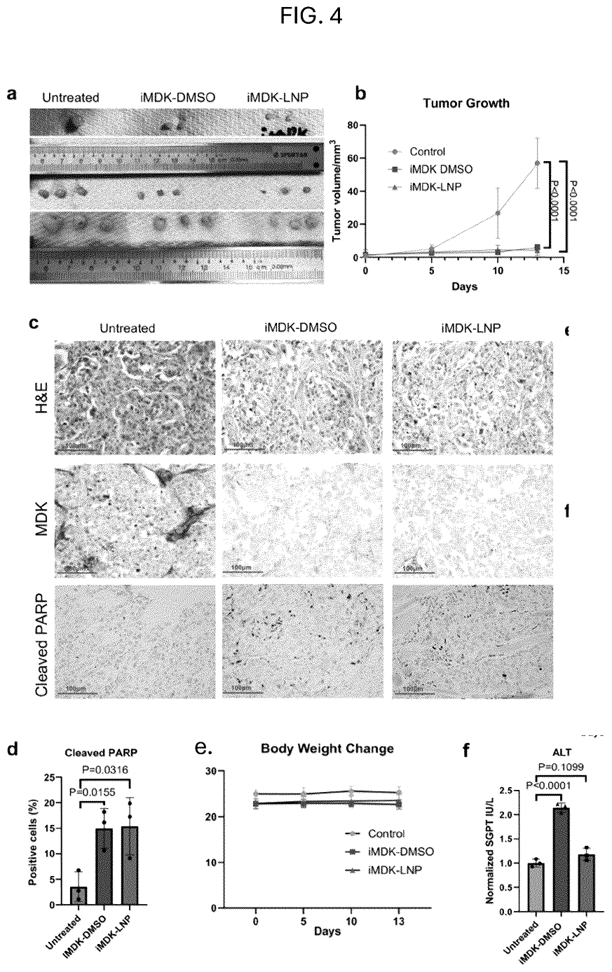

To assess whether LNP[DOTAP] encapsulating iMDK (iMDK-LNP) suppresses growth of H441 cells in vivo, Applicant established an H441 xenograft model using nude mice and treated one group of mice by intraperitoneal (IP) injection with iMDK-LNP formulated in an aqueous carrier and another group of mice with iMDK dissolved in DMSO (iMDK-DMSO). Notably, iMDK-DMSO and iMDK-LNP significantly suppressed the growth of H441 xenografts in both groups compared to untreated H441 xenografts (FIG. 4, panels a and b). MDK protein expression in H441 xenografts from iMDK-DMSO- or iMDK-LNP-treated mice was reduced compared to that from untreated mice (FIG. 4, panel c). Cleaved PARP was significantly induced in H441 xenografts from iMDK-DMSO- or iMDK-LNP-treated mice compared to that from untreated mice (FIG. 4, panels c and d). Importantly, body weight was not affected in iMDK-DMSO-treated mice or iMDK-LNP-treated mice compared to untreated mice (FIG. 4, panel e). The activity of ALT, a marker for liver toxicity, was induced in iMDK-DMSO-treated mice but not in iMDK-LNP-treated mice compared to untreated mice (FIG. 5f). These results indicate that IP-injected iMDK-LNP formulated in an aqueous carrier is as effective as iMDK-DMSO for inducing apoptosis in H441 cells and is less hepatotoxic than iMDK-DMSO in vivo.

### DISCUSSION

iMDK, which was originally identified as a small molecule that represses the expression of MDK, has been shown to suppress the growth of different types of cancers, including KRAS-mutant lung cancers; however, the molecular mechanism by which iMDK suppresses the growth of cancer cells is not fully understood. Here, using RNA-seq combined with a CRISPR/Cas9-mediated gene knockout approach, Applicant determined that iMDK suppresses the growth of H441 cells through induction of the ATF3-CHOP-mediated apoptotic pathway. Using lipid nanoparticle technology, Applicant also developed an iMDK formulation capable of being formulated in an aqueous carrier by encapsulating iMDK in lipid nanoparticles. iMDK in lipid nanoparticles (iMDK-LNP) was as efficient in inducing apoptosis in H441 cells as iMDK in DMSO (iMDK-DMSO), while iMDK-LNP did not cause liver toxicity relative to iMDK-DMSO. These data indicate that iMDK-LNP is a promising and translatable anti-cancer therapy to target subsets of lung cancers that harbor mutant KRAS.

Activation of the ER stress pathway has been considered as a promising approach to tumor growth suppression for the past two decades.^{16,17} For example, an FDA-approved drug, bortezomib, that activates the ER stress pathway to induce apoptosis has been used to treat multiple myeloma and mantle cell lymphoma.¹⁸ Induction of ATF3 was shown to be critical for bortezomib-mediated apoptosis in multiple myeloma,¹⁹ consistent with our data that ATF3 is required for iMDK-mediated apoptosis in H441 papillary lung adenocarcinoma cells. Since iMDK was also shown to induce apoptosis in multiple myeloma cell lines,⁶ it would be worth determining whether iMDK-mediated apoptosis in multiple myeloma is also through the activation of the ATF3-CHOP ER stress pathway. Bortezomib was previously reported to induce apoptosis in A549 cells more efficiently than in H441 cells²⁰ in contrast to our finding that iMDK induces apoptosis in H441 cells but not A549 cells, suggesting that the mechanism of action on ER-mediated apoptosis by iMDK is different from that of bortezomib. This also suggests that iMDK might be useful to treat bortezomib-resistant multiple myeloma.

Therapy for unresectable lung adenocarcinomas has been revolutionized in part thanks to small molecules that bind to mutant driver oncogenes, including mutant KRAS, and suppress tumor growth.²¹ Unfortunately, most lung adenocarcinomas that initially respond to such small molecules recur later, suggesting that a succeeding approach to treat recurrent lung adenocarcinomas is critically needed. For example, the recent discovery of small molecule inhibitors (e.g., sotorasib) that bind to KRAS^{G12C} has revolutionized the treatment of patients with lung cancer that harbor KRAS^{G12C}; however, recurrence by different molecular mechanisms is most likely.⁸ One of the recurrence mechanisms is the activation of the PI3K-AKT pathway, which enables cancer cells that harbor KRAS^{G12C} to evade KRAS^{G12C} inhibition.^{22,23} PI3K-AKT inhibitors were shown to suppress the growth of a subset of sotorasib-resistant cell lines that harbor KRAS^{G12C}.^{22,23}; however, miransertib,¹⁰ a PI3K-AKT inhibitor, failed to induce apoptosis in sotorasib-resistant H358R cells that carry KRAS^{G12C} (FIGS. 1 and 3), indicating that inhibition of the PI3K-AKT pathway is not sufficient to suppress the growth of a portion of KRAS^{G12C} inhibitor-resistant cancer cells. In the present study, Applicant demonstrated that iMDK inhibited the PI3K-AKT pathway as well as induced the ATF3-CHOP ER-mediated apoptotic pathway and in turn caused apoptosis in sotorasib-resistant H358R cells that carry KRAS^{G12C}. Thus, iMDK performs better than PI3K-AKT pathway inhibitors and warrants iMDK for further studies for translation into clinic.

In the present study, Applicant succeeded in encapsulating iMDK, which exhibits low aqueous solubility in its free form, into the LNP formulation DPPC/DOTAP/DSPE-PEG/cholesterol (LNP[DOTAP]) to enable intraperitoneal delivery of iMDK to H441 xenografts without inducing liver toxicity in vivo. This approach has been previously used to enable the hydrophobic small molecule paclitaxel to be administered in an aqueous carrier using albumin-bound nanoparticles. This achievement resulted in nab-paclitaxel (also known as Abraxane), an FDA-approved paclitaxel-derived drug for treating lung and breast cancers with reduced side-toxicity compared to paclitaxel dissolved in polyoxyethylated castor oil (Cremophor EL) and ethanol.²⁴ To our knowledge, the present LNP formulation has not been used for encapsulating hydrophobic small molecules, including iMDK, for preclinical and clinical studies. For hydrophobic small molecules that are not eligible for albumin-bound nanoparticles, the present LNP formulation may be useful to deliver such molecules in vivo, though further pharmacokinetics, pharmacodynamics, and toxicity studies will be necessary.

### CONCLUSION

The present study indicates that iMDK encapsulated in lipid nanoparticles can be administered to mice with reduced excipient-related toxicity relative to iMDK delivered in DMSO. Applicant has shown that iMDK induces ATF3-CHOP ER stress-mediated apoptosis in subsets of human KRAS-mutant lung cancer cells, including KRAS^{G12C} inhibitor-resistant lung cancer cells that do not respond to a PI3K-AKT inhibitor, suggesting that iMDK is a promising therapeutic candidate for treatment of KRAS inhibitor-resistant cancers.

### REFERENCES

1. Hao H, Maeda Y, Fukazawa T, Yamatsuji T, Takaoka M, Bao X H, Matsuoka J, Okui T, Shimo T, Takigawa N, Tomono Y, Nakajima M, Fink-Baldauf I M, Nelson S, Seibel W, Papoian R, Whitsett J A, Naomoto Y. Inhibition of the growth factor MDK/midkine by a novel small molecule compound to treat non-small cell lung cancer. PLoS One. 2013 Aug. 16; 8(8):e71093. doi: 10.1371/journal.pone.0071093. Erratum in: PLoS One. 2024 Jul. 9; 19(7):e0307052. doi: 10.1371/journal.pone.0307052. PMID: 23976985; PMCID: PMC3745462.
2. Masui M, Okui T, Shimo T, Takabatake K, Fukazawa T, Matsumoto K, Kurio N, Ibaragi S, Naomoto Y, Nagatsuka H, Sasaki A. Novel Midkine Inhibitor iMDK Inhibits Tumor Growth and Angiogenesis in Oral Squamous Cell Carcinoma. Anticancer Res. 2016 June; 36(6):2775-81. PMID: 27272788.
3. Erdogan S, Doganlar Z B, Doganlar O, Turkekul K, Serttas R. Inhibition of Midkine Suppresses Prostate Cancer CD133+ Stem Cell Growth and Migration. Am J Med Sci. 2017 September; 354(3):299-309. doi: 10.1016/j.amjms.2017.04.019. Epub 2017 Apr. 27. PMID: 28918838.
4. Shin D H, Jo J Y, Kim S H, Choi M, Han C, Choi B K, Kim S S. Midkine Is a Potential Therapeutic Target of Tumorigenesis, Angiogenesis, and Metastasis in Non-Small Cell Lung Cancer. Cancers (Basel). 2020 Aug. 24; 12(9):2402. doi: 10.3390/cancers12092402. PMID: 32847073; PMCID: PMC7563676.
5. Ueno M, Kariya R, Gunya S, Cheevapruk K, Okada S. Midkine inhibitor (iMDK) induces apoptosis of primary effusion lymphoma via G2/M cell cycle arrest. Leuk Res. 2022 May; 116:106826. doi: 10.1016/j.leukres.2022.106826. Epub 2022 Mar. 12. PMID: 35316715.
6. Cheevapruk K, Ueno M, Sungwan P, Sittithumcharee G, Kariya R, Sampattavanich S, Okada S. Novel Midkine Inhibitor Induces Cell Cycle Arrest and Apoptosis in Multiple Myeloma. Anticancer Res. 2024 March; 44(3):1023-1031. doi: 10.21873/anticanres. 16897. PMID: 38423667.
7. Ishida N, Fukazawa T, Maeda Y, Yamatsuji T, Kato K, Matsumoto K, Shimo T, Takigawa N, Whitsett J A, Naomoto Y. A novel PI3K inhibitor iMDK suppresses non-small cell lung Cancer cooperatively with A MEK inhibitor. Exp Cell Res. 2015 Jul. 15; 335(2):197-206. doi: 10.1016/j.yexcr.2015.03.019. Epub 2015 Apr. 1. PMID: 25839409; PMCID: PMC4485577.
8. Awad M M, Liu S, Rybkin I I, Arbour K C, Dilly J, Zhu V W, Johnson M L, Heist R S, Patil T, Riely G J, Jacobson J O, Yang X, Persky N S, Root D E, Lowder K E, Feng H, Zhang S S, Haigis K M, Hung Y P, Sholl L M, Wolpin B M, Wiese J, Christiansen J, Lee J, Schrock A B, Lim L P, Garg K, Li M, Engstrom L D, Waters L, Lawson J D, Olson P, Lito P, Ou S I, Christensen J G, Jänne P A, Aguirre A J. Acquired Resistance to KRASG12C Inhibition in Cancer. N Engl J Med. 2021 Jun. 24; 384(25):2382-2393. doi: 10.1056/NEJMoa2105281. PMID: 34161704; PMCID: PMC8864540.
9. Zhao Y, Murciano-Goroff Y R, Xue J Y, Ang A, Lucas J, Mai T T, Da Cruz Paula A F, Saiki A Y, Mohn D, Achanta P, Sisk A E, Arora K S, Roy R S, Kim D, Li C, Lim L P, Li M, Bahr A, Loomis B R, de Stanchina E, Reis-Filho J S, Weigelt B, Berger M, Riely G, Arbour K C, Lipford J R, Li B T, Lito P. Diverse alterations associated with resistance to KRAS(G12C) inhibition. Nature. 2021 November; 599(7886):679-683. doi: 10.1038/s41586-021-04065-2. Epub 2021 Nov. 10. PMID: 34759319; PMCID: PMC8887821.
10. Lapierre J M, Eathiraj S, Vensel D, Liu Y, Bull C O, Cornell-Kennon S, Iimura S, Kelleher E W, Kizer D E, Koerner S, Makhija S, Matsuda A, Moussa M, Namdev N, Savage R E, Szwaya J, Volckova E, Westlund N, Wu H, Schwartz B. Discovery of 3-(3-(4-(1-Aminocyclobutyl)phenyl)-5-phenyl-3H-imidazo[4,5-b]pyridin-2-yl)pyridin-2-amine (ARQ 092): An Orally Bioavailable, Selective, and Potent Allosteric AKT Inhibitor. J Med Chem. 2016 Jul. 14; 59(13):6455-69. doi: 10.1021/acs.jmedchem.6b00619. Epub 2016 Jun. 29. PMID: 27305487.
11. Chen J, Bardes E E, Aronow B J, Jegga A G. ToppGene Suite for gene list enrichment analysis and candidate gene prioritization. Nucleic Acids Res. 2009 July; 37 (Web Server issue): W305-11. doi: 10.1093/nar/gkp427. Epub 2009 May 22. PMID: 19465376; PMCID: PMC2703978.
12. Jiang H Y, Wek S A, McGrath B C, Lu D, Hai T, Harding H P, Wang X, Ron D, Cavener D R, Wek R C. Activating transcription factor 3 is integral to the eukaryotic initiation factor 2 kinase stress response. Mol Cell Biol. 2004 February; 24(3):1365-77. doi: 10.1128/MCB.24.3.1365-1377.2004. PMID: 14729979; PMCID: PMC321431.
13. Micheau O, Tschopp J. Induction of TNF receptor I-mediated apoptosis via two sequential signaling complexes. Cell. 2003 Jul. 25; 114(2):181-90. doi: 10.1016/s0092-8674 (03) 00521-x. PMID: 12887920.
14. Nakajima E C, Drezner N, Li X, Mishra-Kalyani P S, Liu Y, Zhao H, Bi Y, Liu J, Rahman A, Wearne E, Ojofeitimi I, Hotaki L T, Spillman D, Pazdur R, Beaver J A, Singh H. FDA Approval Summary: Sotorasib for KRAS G12C-Mutated Metastatic NSCLC. Clin Cancer Res. 2022 Apr. 14; 28(8):1482-1486. doi: 10.1158/1078-0432.CCR-21-3074. PMID: 34903582; PMCID: PMC9012672.
15. Verma A, Stellacci F. Effect of surface properties on nanoparticle-cell interactions. Small. 2010 January; 6(1):12-21. doi: 10.1002/smll.200901158. PMID: 19844908.
16. Kim I, Xu W, Reed J C. Cell death and endoplasmic reticulum stress: disease relevance and therapeutic opportunities. Nat Rev Drug Discov. 2008 December; 7(12):1013-30. doi: 10.1038/nrd2755. PMID: 19043451.
17. Schönthal A H. Pharmacological targeting of endoplasmic reticulum stress signaling in cancer. Biochem Pharmacol. 2013 Mar. 1; 85(5):653-666. doi: 10.1016/j.bcp.2012.09.012. Epub 2012 Sep. 20. PMID: 23000916.
18. Richardson P G, Sonneveld P, Schuster M W, Irwin D, Stadtmauer E A, Facon T, Harousseau J L, Ben-Yehuda D, Lonial S, Goldschmidt H, Reece D, San-Miguel J F, Bladé J, Boccadoro M, Cavenagh J, Dalton W S, Boral A L, Esseltine D L, Porter J B, Schenkein D, Anderson K C; Assessment of Proteasome Inhibition for Extending Remissions (APEX) Investigators. Bortezomib or high-dose dexamethasone for relapsed multiple myeloma. N Engl J Med. 2005 Jun. 16; 352(24):2487-98. doi: 10.1056/NEJMoa043445. PMID: 15958804.
18. Narita T, Ri M, Masaki A, Mori F, Ito A, Kusumoto S, Ishida T, Komatsu H, Iida S. Lower expression of activating transcription factors 3 and 4 correlates with shorter progression-free survival in multiple myeloma patients receiving bortezomib plus dexamethasone therapy. Blood Cancer J. 2015 Dec. 4; 5(12):e373. doi: 10.1038/bcj.2015.98. PMID: 26636288; PMCID: PMC4735074.
20. Li Y, Dong S, Tamaskar A, Wang H, Zhao J, Ma H, Zhao Y. Proteasome Inhibitors Diminish c-Met Expression and Induce Cell Death in Non-Small Cell Lung Cancer Cells. Oncol Res. 2020 Dec. 10; 28(5):497-507. doi: 10.3727/096504020X15929939001042. Epub 2020 Jun. 24. PMID: 32580819; PMCID: PMC7751221.
21. Tan A C, Tan D S W. Targeted Therapies for Lung Cancer Patients With Oncogenic Driver Molecular Alterations. J Clin Oncol. 2022 Feb. 20; 40(6):611-625. doi: 10.1200/JCO.21.01626. Epub 2022 Jan. 5. PMID: 34985916.
22. Misale S, Fatherree J P, Cortez E, Li C, Bilton S, Timonina D, Myers D T, Lee D, Gomez-Caraballo M, Greenberg M, Nangia V, Greninger P, Egan R K, McClanaghan J, Stein G T, Murchie E, Zarrinkar P P, Janes M R, Li L S, Liu Y, Hata A N, Benes C H. KRAS G12C NSCLC Models Are Sensitive to Direct Targeting of KRAS in Combination with PI3K Inhibition. Clin Cancer Res. 2019 Jan. 15; 25(2):796-807. doi: 10.1158/1078-0432.CCR-18-0368. Epub 2018 Oct. 16. PMID: 30327306.
23. Chan C H, Chiou L W, Lee T Y, Liu Y R, Hsieh T H, Yang C Y, Jeng Y M. PAK and PI3K pathway activation confers resistance to KRASG12C inhibitor sotorasib. Br J Cancer. 2023 January; 128(1):148-159. doi: 10.1038/s41416-022-02032-w. Epub 2022 Nov. 1. PMID: 36319849; PMCID: PMC9814377.
24. Ma P, Mumper R J. Paclitaxel Nano-Delivery Systems: A Comprehensive Review. J Nanomed Nanotechnol. 2013 Feb. 18; 4(2):1000164. doi: 10.4172/2157-7439.1000164. PMID: 24163786; PMCID: PMC3806207.
25. Tomoshige K, Stuart W D, Fink-Baldauf I M, Ito M, Tsuchiya T, Nagayasu T, Yamatsuji T, Okada M, Fukazawa T, Guo M, Maeda Y. FOXA2 Cooperates with Mutant KRAS to Drive Invasive Mucinous Adenocarcinoma of the Lung. Cancer Res. 2023 May 2; 83(9):1443-1458. doi: 10.1158/0008-5472.CAN-22-2805. PMID: 37067057; PMCID: PMC10160002.
26. Love M I, Huber W, Anders S. Moderated estimation of fold change and dispersion for RNA-seq data with DESeq2. Genome Biol. 2014; 15(12):550. doi: 10.1186/s13059-014-0550-8. PMID: 25516281; PMCID: PMC4302049.
27. Dunn A W, Kalinichenko V V, Shi D. Highly Efficient In Vivo Targeting of the Pulmonary Endothelium Using Novel Modifications of Polyethylenimine: An Importance of Charge. Adv Healthc Mater. 2018 December; 7(23):e1800876. doi: 10.1002/adhm.201800876. Epub 2018 Nov. 6. PMID: 30398703.

All percentages and ratios are calculated by weight unless otherwise indicated.

All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm.”

Every document cited herein, including any cross referenced or related patent or application, is hereby incorporated herein by reference in its entirety unless expressly excluded or otherwise limited. All accessioned information (e.g., as identified by PUBMED, PUBCHEM, NCBI, UNIPROT, or EBI accession numbers) and publications in their entireties are incorporated into this disclosure by reference in order to more fully describe the state of the art as known to those skilled therein as of the date of this disclosure. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications may be made without departing from the spirit and scope of the invention. It is therefore intended to cover in the appended claims all such changes and modifications that are within the scope of this invention.

### EXEMPLARY COMBINATIONS

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A method of treating a cancer in an individual in need thereof, comprising administering a therapeutically effective amount of iMDK (3-[2-[(4-Fluorophenyl)methyl]imidazo[2,1-b]thiazol-6-yl]-2H-1-benzopyran-2-one) encapsulated in a water-soluble lipid nanoparticle (LNP) to the individual.

### Example 2

The method of Example 1, wherein the LNP comprises 1,2-dioleoyl-3-trimethylammonium-propane (DOTAP).

### Example 3

The method of any one or more of Examples 1-2, wherein the LNP further comprises dipalmitoylphosphatidylcholine (DPPC), distearoylphosphatidylethanolamine-polyethylene glycol (DSPE-PEG), and cholesterol.

### Example 4

The method of any one or more of Examples 1-3, wherein DPPC, DOTAP, DSPE-PEG, and cholesterol are present in a weight ratio of about 10:5:5:2.

### Example 5

The method of any one or more of Examples 1-4, wherein the cancer retains the ATF3-CHOP-mediated apoptotic pathway.

### Example 6

The method of any one or more of Examples 1-5, wherein the cancer is a KRAS-mutant cancer.

### Example 7

The method of any one or more of Examples 1-6, wherein the cancer is a KRAS-mutant lung cancer.

### Example 8

The method of any one or more of Examples 1-7, wherein the lung cancer is characterized by the driver oncogene KRAS^{G12V}.

### Example 9

The method of any one or more of Examples 1-8, wherein the lung cancer is a papillary lung adenocarcinoma.

### Example 10

The method of any one or more of Examples 1-7, wherein the lung cancer is characterized by the driver oncogene KRAS^{G12C}.

### Example 11

The method of any one or more of Examples 1-7 or 10, wherein the lung cancer is bronchioalveolar carcinoma.

### Example 12

The method of any one or more of Examples 1-7 or 10, wherein the lung cancer is non-small cell lung cancer (NSCLC).

### Example 13

The method of any one or more of Examples 1-12, wherein the cancer is resistant to a KRAS^{G12C} inhibitor.

### Example 14

The method of any one or more of Examples 1-13, wherein the KRAS^{G12C} inhibitor is sotorasib.

### Example 15

The method of any one or more of Examples 1-14, wherein the cancer does not respond to inhibition of the PI3K-AKT pathway.

### Example 16

The method of any one or more of Examples 1-15, wherein the cancer is selected from oral squamous cell carcinoma, prostate cancer, primary effusion lymphoma, multiple myeloma, and combinations thereof.

### Example 17

The method of any one or more of Examples 1-16, wherein the administration is via a route selected from intraperitoneal, intravenous, subcutaneous, intramuscular, inhalation, oral, topical, and combinations thereof.

### Example 18

The method of any one or more of Examples 1-17, wherein the administration results in reduced hepatotoxicity as compared to administration of iMDK dissolved in dimethyl sulfoxide (DMSO).

### Example 19

A method of identifying an individual likely to respond to iMDK therapy and treating the individual, comprising: (a) determining that the individual has a cancer that retains the ATF3-CHOP-mediated apoptotic pathway; and (b) administering to the individual a therapeutically effective amount of iMDK encapsulated in a water-soluble lipid nanoparticle (LNP).

### Example 20

The method of Example 19, wherein the cancer is a KRAS-mutant cancer that is resistant to a KRAS^{G12C} inhibitor and does not respond to PI3K-AKT inhibition.

### Example 21

A composition comprising iMDK (3-[2-[(4-Fluorophenyl)methyl]imidazo[2,1-b]thiazol-6-yl]-2H-1-benzopyran-2-one) encapsulated in a water-soluble lipid nanoparticle (LNP) comprising 1,2-dioleoyl-3-trimethylammonium-propane (DOTAP).

### Example 22

The composition of Example 21, wherein the LNP further comprises dipalmitoylphosphatidylcholine (DPPC), distearoylphosphatidylethanolamine-polyethylene glycol (DSPE-PEG), and cholesterol.

### Example 23

The composition of any one or more of Examples 21-22, wherein DPPC, DOTAP, DSPE-PEG, and cholesterol are present in a weight ratio of about 10:5:5:2.

### Example 24

The composition of any one or more of Examples 21-23, wherein the LNP has a particle size of about 100 nm to about 300 nm, or a particle size of about 180 to about 220 nm, or about 190 to about 210 nm, or about 200 nm, or wherein at least 90% of the LNPs in the composition have a particle size of a particle size of about 180 to about 220 nm, or about 190 to about 210 nm, or about 200 nm.

### Example 25

The composition of any one or more of Examples 21-24, wherein the LNP has a particle size of about 150 nm to about 250 nm.

### Example 26

A method for making a lipid nanoparticle (LNP) comprising iMDK (3-[2-[(4-Fluorophenyl)methyl]imidazo[2,1-b]thiazol-6-yl]-2H-1-benzopyran-2-one), the method comprising: (a) dissolving dipalmitoylphosphatidylcholine (DPPC), 1,2-dioleoyl-3-trimethylammonium-propane (DOTAP), distearoylphosphatidylethanolamine-polyethylene glycol (DSPE-PEG), and cholesterol in a first organic solvent to form a lipid solution; (b) removing the first organic solvent to form a lipid film; (c) hydrating the lipid film with an aqueous medium to form a liposome suspension; (d) sonicating the liposome suspension to reduce particle size; (e) adding iMDK dissolved in a second organic solvent to the liposome suspension under sonication; and (f) purifying the resulting suspension by removing unencapsulated iMDK and residual second organic solvent.

What is claimed is:

1. A method of treating a cancer in an individual in need thereof, comprising administering a therapeutically effective amount of a hydrophobic small molecule encapsulated in a water-soluble lipid nanoparticle (LNP) to the individual.

2. The method of claim 1, wherein the LNP comprises 1,2-dioleoyl-3-trimethylammonium-propane (DOTAP), dipalmitoylphosphatidylcholine (DPPC), distearoylphosphatidylethanolamine-polyethylene glycol (DSPE-PEG), and cholesterol.

3. The method of claim 2, wherein the LNP is in an aqueous carrier.

4. The method of claim 3, wherein DPPC, DOTAP, DSPE-PEG, and cholesterol are present in a weight ratio of about 10:5:5:2.

5. The method of claim 1, wherein the cancer retains the ATF3-CHOP-mediated apoptotic pathway.

6. The method of claim 1, wherein the cancer is a KRAS-mutant cancer.

7. The method of claim 6, wherein the cancer is a KRAS-mutant lung cancer.

8. The method of claim 7, wherein the lung cancer is characterized by the driver oncogene KRAS^{G12V}.

9. The method of claim 8, wherein the lung cancer is a papillary lung adenocarcinoma.

10. The method of claim 7, wherein the lung cancer is characterized by the driver oncogene KRAS^{G12C}.

11. The method of claim 10, wherein the lung cancer is bronchioalveolar carcinoma.

12. The method of claim 10, wherein the lung cancer is non-small cell lung cancer (NSCLC).

13. The method of claim 1, wherein the cancer is resistant to a KRAS^{G12C} inhibitor.

14. The method of claim 13, wherein the KRAS^{G12C} inhibitor is sotorasib.

15. The method of claim 1, wherein the cancer does not respond to inhibition of the PI3K-AKT pathway.

16. The method of claim 1, wherein the cancer is selected from oral squamous cell carcinoma, prostate cancer, primary effusion lymphoma, multiple myeloma, and combinations thereof.

17. The method of claim 1, wherein the administration is via a route selected from intraperitoneal, intravenous, subcutaneous, intramuscular, inhalation, oral, topical, and combinations thereof.

18. The method of claim 1, wherein the hydrophobic small molecule is iMDK (3-[2-[(4-Fluorophenyl)methyl]imidazo[2,1-b]thiazol-6-yl]-2H-1-benzopyran-2-one)

19. A method of identifying an individual likely to respond to iMDK therapy and treating the individual, comprising: (a) determining that the individual has a cancer that retains the ATF3-CHOP-mediated apoptotic pathway; and (b) administering to the individual a therapeutically effective amount of iMDK encapsulated in a water-soluble lipid nanoparticle (LNP).

20. A composition comprising iMDK (3-[2-[(4-Fluorophenyl)methyl]imidazo[2,1-b]thiazol-6-yl]-2H-1-benzopyran-2-one) encapsulated in a water-soluble lipid nanoparticle (LNP) comprising 1,2-dioleoyl-3-trimethylammonium-propane (DOTAP) dipalmitoylphosphatidylcholine (DPPC), distearoylphosphatidylethanolamine-polyethylene glycol (DSPE-PEG), and cholesterol.

21. The composition of claim 20, wherein the LNP is provided in an aqueous carrier.
